(19) United States
(12) Patent Application Publication
(10) Pub. No.: US 2006/0224072 A1
(43) Pub. Date: Oct. 5, 2006

(54) Disposable extended wear heart monitor patch

(75) Inventor: Adnan Shennib, Dublin, CA (US)

Correspondence Address: DONALD R. GREENE, P.O. BOX 6238, GOODYEAR, AZ, 85338, US

(73) Assignee: CardioVu, Inc., Dublin, CA (US)

(21) Appl. No.: 11/095,821

(22) Filed: Mar. 31, 2005

(51) Int. Cl. A61B 5/04

(52) U.S. Cl. 600/509

(57) ABSTRACT

The invention provides a disposable sensor patch for non-invasive monitoring and recording of infrequent cardiac events. The patch is thin and flexible for comfortable wear on the person's chest for automatic analysis and recording of ECG. The patch is inexpensive and simple for self-administration. The patch incorporates a battery, ECG amplifier, and a processor for analyzing ECG waveform and recording events. A software algorithm searches for a cardiac abnormality. The patch is designed for continuous long-term wear exceeding 3 days for diagnostic monitoring and exceeding 14 days for event detection. In one embodiment a preformatted report is automatically generated by the patch for wireless transmission to a reporting device such as a generic printer or a wireless network system. The patch may also incorporate a marker switch to correlate recorded ECG data with the patient's perception of a cardiac event.

### CROSS REFERENCE TO RELATED APPLICATIONS

This application is related to co-pending patent application Ser. No. 10/913,586 and Ser. No. 10,913,166, filed jointly Aug. 5, 2004. These applications are incorporated herein in their entirety by the reference thereto.

### BACKGROUND OF THE INVENTION

1. Technical Field

The invention relates to electrocardiogram (ECG) detection. More particularly, this invention relates to non-invasive monitoring and detection of heart abnormalities.

2. Description of the Prior Art

Cardiovascular diseases are pervasive, contributing to over 2.4 million deaths annually in the United States alone. Patients suffering from heart disease often have no symptoms until a heart attack develops. Other symptoms are intermittent and often ignored after the resolution of the cardiac event. Delay in recognition and treatment of a heart disease leads to more damage to the heart, higher cost of hospitalization and lower quality of life for the survivors.

Certain heart abnormalities may be detected by standard ECG equipment available in hospitals and clinical settings. However, many other abnormalities are so intermittent and cannot be detected with typical in-clinic evaluations. Certain arrhythmias occur only a few times followed by a fatal heart attack. Syncope, or temporary loss of consciousness, is another common problem accounting for 3 percent of emergency room visits according to hospital reports. These cardiac abnormalities are illusive and may not be detected with current ECG systems, including Holter and cardiac event monitors.

Holter monitors are used for ambulatory monitoring and recording of ECG. These instruments typically use 5 or more ECG electrodes attached to the chest at one end and connected to a portable device at the other end. The electronic device is worn or strapped to the body and records ECG signals in its memory. Holter monitors may also incorporate an alarm to warn the patient of an adverse cardiac event. After typically 24 or 48 hours of monitoring, the Holter monitor is returned to the clinic, where the recorded ECG data are downloaded for review, record keeping, and for further analysis. Sizable memory is typically required to record many hours of continuous ECG.

Cardiac event monitors record a few minutes of ECG that occur during an intermittent cardiac event, i.e. heart palpitation, dizziness, syncope, chest pain, etc. There are generally two types of cardiac event monitors; (1) post-symptom event recorder and (2) looping memory (pre-symptom) recorder. The postsymptom event recorder is simple to use and may be handheld and applied by the patient on the chest upon the occurrence of a cardiac event. Typically, the patient presses the electrode feet of the monitor to the chest and activates the monitor's record button to begin recoding the on-going cardiac event. The metal feet allow conduction of the ECG signal into the monitor's memory. Since the recording is post-symptomatic, the cardiac events may be missed if too short or if there is a delay in the response by the patient.

The looping memory event recorder resolves the delay issue by pre-attaching the monitor to the patient's chest via two electrodes for the duration of monitoring. These devices therefore continuously monitor the ECG and have the ability to retain the most recent segment of the ECG just prior to the activation of the recording switch. For example, when a patient experiences a palpitation, the device keeps in memory the prior 45 seconds of ECG as well as 15 seconds post the activation. With this method of monitoring, many transitory symptoms can be documented. A patient with symptoms of syncope would be given this type of monitor programmed to record several minutes before activation. This is to allow sufficient time for the person to recover from a faint episode, which may last several minutes.

These monitors may have a display to inform the patient if and how many events have been recorded and the status of the battery life. Long-term cardiac monitoring is typically 30 days, although some patients with less frequent symptoms may be monitored for several months.

Holter monitors are generally more diagnostic than event recorders but are limited to 1 or 2 days of recording. Event recorders are less bulky and more comfortable to wear but lack the diagnostic capabilities of Holter monitors. Daily or periodic trans-telephonic data transmission of ECG data is employed for individuals who require long term monitoring of their ECG. The patches (electrodes) employed with these monitors are disposable. However, the electronic base unit is reusable as it is loaned to patients as part of the diagnostic service provided by the clinic.

The discomfort of wear and inconvenience of current long term monitors have led to the development of implants such as the Reveal® Insertable Loop Recorder, marketed by Medtronic of Minneapolis, Minn. Although more convenient and can be implanted for up to 14 months, the cost and risk of surgery limits the application of these monitoring devices for most persons with potential cardiac cases.

Holter monitors, event recorders and implant monitors are ambulatory ECG monitors that are not only expensive but also require additional specialized instruments comprising hardware and software to retrieve, store, and produce patient reports. The cost and training requirement for these ambulatory ECG systems presents a major barrier for individuals and many in the medical community, particularly those not specializing in cardiac care. The above prior art instruments and methods and others discussed below fall short of providing low cost effective long term cardiac monitoring.

U.S. patent application serial no. 2003/0069510 to Semler discloses a disposable vital signs monitor in the form of a patch that is a “flexible, nominally flat planer form having integral gel electrodes, a sticky-back rear surface, an internal flex circuit capable of sensing, recording, and play out several minutes of the most recently acquired ECG waveform data and a front surface that includes an output port preferably having one or more snap connectors compatible with lead harness . . . .” The monitor disclosed by Semler is designed for short term applications as stated: “a relatively short term battery life, as it is intended for limited-term use.” This and other limitations render Semler's invention inadequate for long-term assessment of intermittent cardiac abnormalities.

U.S. Pat. No. 5,634,468 to Platt et al. discloses a sensor patch for obtaining physiologic data, including temperature, and transmitting a conditioned signal to a nearby portable unit and subsequently to a remote monitoring equipment. In cardiac applications, Platt's patch neither saves ECG data nor performs ECG analysis for detecting cardiac abnormalities. For these purposes, it relies on external devices as disclosed.

U.S. patent application serial no. 2003/0083559 to Thompson discloses a peripheral monitor patch for attachment to a patient including high capacity memory for storage and later retrieval of the sensed ECG data. The patch comprises non-contact electrodes. The disclosed patch neither provides diagnostic capability nor means for long term wear and monitoring.

It would be advantageous to provide an inexpensive non-invasive long-term heart monitor for detecting intermittent cardiac abnormalities such as arrhythmias and syncope. Furthermore, this monitor would be extremely comfortable to wear.

It would also be advantageous to provide a diagnostic multi-lead ECG monitor suitable for long term-wear exceeding the 24-48 hours provided by standard Holter monitors.

It would also be desirable to provide an alternative to ECG event recorders whereby the device is comfortably and continuously worn for at least 14 days and preferably exceeding 30 days targeting detection of rare cardiac events.

It would also be desirable to provide a non-obtrusive body worn monitor for continuous long-term wear, including during bathing and swimming.

It would also be advantageous to provide an ECG monitor with built-in report generation capability and wireless transmission of reports such that a cardiac report can be obtained using a generic printer or wireless network. This eliminates the need for specialized personnel or monitoring instruments.

### SUMMARY OF THE INVENTION

The invention provides a disposable sensor patch for non-invasive monitoring and recording of intermittent cardiac events. The patch is thin and flexible for comfortable wear on the person's chest for analysis and recording of ECG signals present on the surface of the body. The self-adhered patch is inexpensive and simple for self-administration. The patch incorporates a battery, ECG amplifier, and a processor for analyzing ECG waveform and recording events. A software algorithm searches for a cardiac abnormality and records the corresponding ECG segment. The patch is designed for continuous long-term wear. In a preferred embodiment, a preformatted report is automatically generated by the patch and transmitted wirelessly to a generic reporting device such as a printer or a wireless network system using infrared or RF signals. The patch may also incorporate a marker switch to correlate recorded ECG data with the patient's perception of a cardiac event. Because of the extreme comfort of wear, a multi-lead configuration can be worn for periods exceeding 48 hours thus providing improved diagnostics when compared to standard Holter monitors. A single lead configuration, targeting arrhythmia detection, can be worn for at least 14 days and preferably 30 days or more, thus providing a non-invasive alternative to standard event recorders and implant monitors. The patch is preferably treated with medication material to minimize possible contamination and infections of the skin since it is worn for extended periods. The medication may include anti-bacterial, anti-microbial and like agents such as zinc oxide.

Unlike conventional Holter monitors or event recorders, the invented patch is totally unobtrusive, comfortable to wear and waterproof for continuous uninterrupted wear, even during bathing. The patch may also include an indicator for alerting the user of a detected event. This allows the user with suspected heart abnormality to wear several patches sequentially until a cardiac event is detected and recorded by the patch.

### BRIEF DESCRIPTION OF THE DRAWINGS

FIG. 1 is a view of the extended wear ECG monitor patch placed on the chest of a person;

FIG. 2 is a top view of the extended wear ECG monitor patch having 3 electrodes, flexible circuit, battery, recording switch and other major components;

FIG. 3 is a cross section view of the patch of FIG. 2, showing the various layers with thickness exaggerated for clarity;

FIG. 4 shows a two-electrode band-shaped embodiment;

FIG. 5 shows an embodiment of the ECG patch with 4 electrodes and an LCD indicator;

FIG. 6 shows a C-shaped multi-lead diagnostic embodiment of the patch;

FIG. 7 shows the C-patch diagnostic embodiment of FIG. 6 placed on the chest and encompassing the left breast of a female;

FIG. 8 shows optical transmission of a preformatted ECG report to a printer device.

FIG. 9 shows wireless transmission of ECG data, recorded by the patch to a computer via a receiver wand.

### DETAILED DESCRIPTION OF THE INVENTION

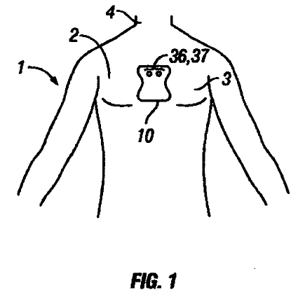

The invention, shown in various embodiments of FIGS. 1-9 is a disposable non-invasive patch for detection of intermittent cardiac abnormalities. The patch 10 is thin, flat, and flexible for placement on the chest area 2 of a person 1 whose heart is being examined for possible abnormality. The sensor patch relies on a surface electrocardiogram (ECG) for detecting and analyzing non-invasively the electrical activity of the heart. The smart patch is fully self-contained and self-powered. The patch analyzes the ECG for an extended period of time depending on the application. Patterns of ECG abnormalities are recorded automatically and a report is generated by the patch and transmitted to a reporting device directly. The sensor patch is low cost for disposable applications and self-administration.

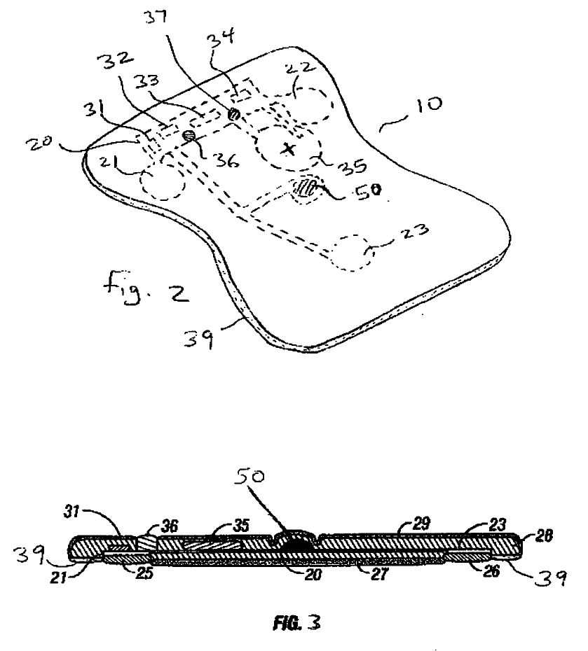

Referring to the embodiment of FIGS. 2 and 3, the sensor patch 10 comprises three ECG electrodes 21, 22, and 23, an ECG amplifier 31, a processor 33, and a battery 35. The processor 33 is typically a microprocessor or a digital signal processor for performing numerical computation on data obtained from an analog-to-digital converter 32. The sensor patch 10 also incorporates a memory 34, referring generally here to all types of solid-state memory for storage of program data and acquired ECG data. A record switch 50 allows the user to record a cardiac event whenever felt.

The electronic assembly of the patch is formed of a flexible circuit substrate 20 with trace extensions to the electrodes 21, 22, 23, and to the battery 35. Conductive gel 25, 26 covers the electrodes 21, 22, respectively, as well as the other electrode not shown in the view of FIG. 3. The conductive gel 25 and 26 contacts the person's skin directly to conduct surface ECG potentials to the electrodes and subsequently to the ECG amplifier 31. The electrodes may be pre-gelled as shown or alternately made for dry contact (not shown) with electrodes directly contacting the skin. A non-conductive pad 27 electrically separates the electrodes and may comprise an adhesive gel, i.e. Hydrogel, for enhancing adhesion of the patch 10 to the skin. The non-conductive pad 27 may also be made of soft low-durometer rubber or elastomeric material. The patch 10 also comprises a thin substrate 28 for providing structural support. The substrate 28 is made of soft flexible sheath material, such as polyurethane, cotton, cloth or made from the same material as the pad 27. The thickness of the patch device 10 (not shown to scale for clarity) is preferably in the range of 1.5 and 2.5 mm, but preferably no more than 3 mm.

Non-conductive waterproof adhesive 39 present at the perimeter of the interior side of the patch prevents water entry and provides long term adhesion to the skin. The waterproof skin adhesive 39 prevents contamination of electrodes thus maintaining long-term integrity of the skin-electrode electrical conductivity. This is critical for providing long term function of the monitor patch while allowing the user to be exposed to water such as during bathing and swimming. The substrate 28, adhesive 39 and other materials used in the design of the patch are preferably air permeable with respect to the skin in order to prevent moisture accumulation and contamination due to perspiration. Anti-microbial and anti-bacterial agents are preferably incorporated in the design of the patch, particularly at the skin contact areas, to prevent contamination of the patch and infection of the skin during the extended wear of the device. In the preferred embodiments, the patch is self-adhered. A porous and/or air permeable waterproof cover 29 protects the outer surface of the patch from external water exposure while allowing drying of the skin.

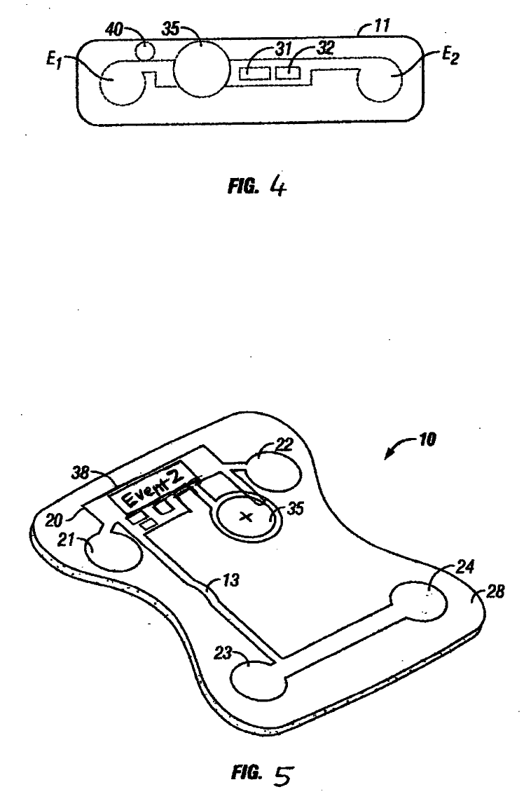

In the embodiments of FIGS. 2-3, the extended wear heart monitor patch 10 comprises three ECG electrodes for placement on the heart area 3 as shown in FIG. 1. The electrodes are arranged to provide a modified three-lead configuration with the electrodes 21, 22, 23 representing right arm (RA), left arm (LA) and left leg (LL) leads as in standard ECG instrumentation. This configuration results in standard, direct lead measurements Lead-I, Lead-II, Lead-III. Other electrode placements and lead configurations are possible. For example, FIG. 4- shows a band-shaped patch 11 with a two-electrode embodiment, E₁ and E₂, for sensing the surface ECG. A multi-color LED 40 is used to indicate heart activity and event detection.

The invented patch is particularly suited to detect infrequent and rare events such as atrial fibrillation and syncope. These events often elude conventional ECG instruments. Since the invented patch is waterproof and can be worn continuously, even during showering and swimming, cardiac events are readily detected and documented. The detection occurs automatically and optionally manually. Automatic detection and recording occurs by continuously monitoring and analyzing ECG data by the processor 33. Manually recording is provided by an optional switch 50, which is activated when the patient becomes aware of a cardiac episode. The activation of the switch 50 triggers a recording session of a predetermined length, for example 3 minutes prior activation plus 2 minutes post activation. This method ensures detection and recording of even the most transient episodes such as syncope, which is accompanied by a temporary loss of consciousness.

Real-time ECG analysis in the invention performed by the processor 33 allows for automatic detection of cardiac abnormalities. These events can be detected by comparing the characteristics of sensed ECG with predetermined limits and patterns. For example, shifts in certain segments of the ECG, such as the ST-segment, QT interval and QRS width, can be used to determine and record a cardiac event. By focusing on recording mostly cardiac events, memory size is reduced for producing smaller and more wearable device than those of conventional monitors.

The detection of a heart abnormality is indicated by a optional indicator. In the embodiment shown in FIGS. 1-3, a light emitting diode (LED) indicator 36 is provided. The indictor many be multi-colored to indicate different levels of indication. For example, a blinking green LED light can indicate a normal heart function and while a red LED light indicates a cardiac event condition. The LED can also be used to indicate proper path operation during the collection of ECG data. For example, the LED can be flashing in synchrony with QRS pulses upon proper placement of the smart patch and upon detection of ECG signals.

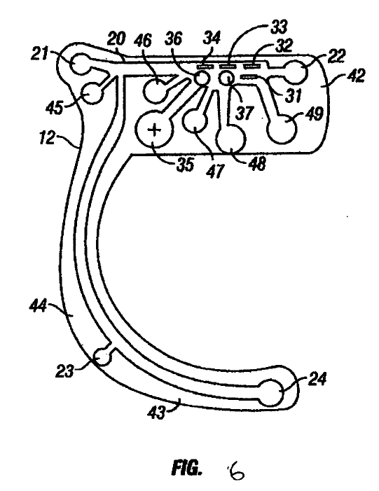

Other possible indicators include audible transducers, such as a buzzer (not shown) or a speaker (not shown; and other visual indicator types, such as a liquid crystal display (LCD) 38 as shown in FIG. 5. The advantage of an LCD indicator is to communicate more clearly the operation of the patch and condition detected. A key feature of the invention in the preferred embodiment is integrating in a single low cost patch the combination of ECG analysis and detection of cardiac events. FIG. 5 shows a 4-electrode embodiment of the patch including a right leg (RL) electrode.

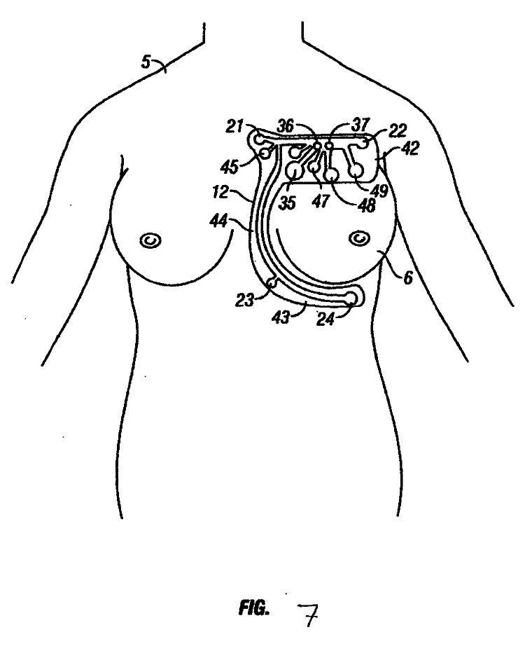

FIGS. 6 & 7 show a nine-electrode patch 12 arranged in a “C” configuration. The electrodes are arranged to obtain modified twelve-lead measurements, excluding the V6 lead. This and other multi-lead configurations provide multi-axis or vectorcardiograph capability for improved diagnostics. The electrodes 21, 22, 23, 24 offer bipolar frontal plane ECG (lead-I, II, and III) while electrodes 45, 46, 47, 48, and 49 offer unipolar precordial ECG, generally representing the horizontal plane, for leads V1, V2, V3, V4, and V5, respectively. The “C” patch encompasses the left breast 6 having an upper segment 42, lower segment 43, and sternum segment 44. The “C” patch 12 is particularly suitable for fitting on a female 5 as shown in FIG. 7.

These and other electrode configurations are possible, as will become obvious to those skilled in the art of ECG measurements. Because the electrodes are integrated within the patch of the invention, motion artifact is significantly reduced when compared to standard ECG with separate electrodes and cabling. Furthermore, the integrated patch allows for inconspicuous, convenient long-term ambulatory applications.

Multi-lead patch configurations are particularly suited for diagnostic monitoring extended beyond 24 to 48 hours offered by conventional Holter monitors. This is possible by the present invention for at least three reasons. First, the invented patch is flexible and more comfortable to wear. Second, there is no need for large memory used for continuous recording in Holter monitors, since only relevant ECG data is recorded. Third, the patch is waterproof thus can be worn continuously without removal.

Signal processing by processor 33 is particularly suited for performing signal averaging to enhance certain details of the sensed ECG. Signal-averaged ECG involves the averaging of a large number of ECG periods, particularly for QRS, ST or QT segments, to enhance the detection of small fluctuations.

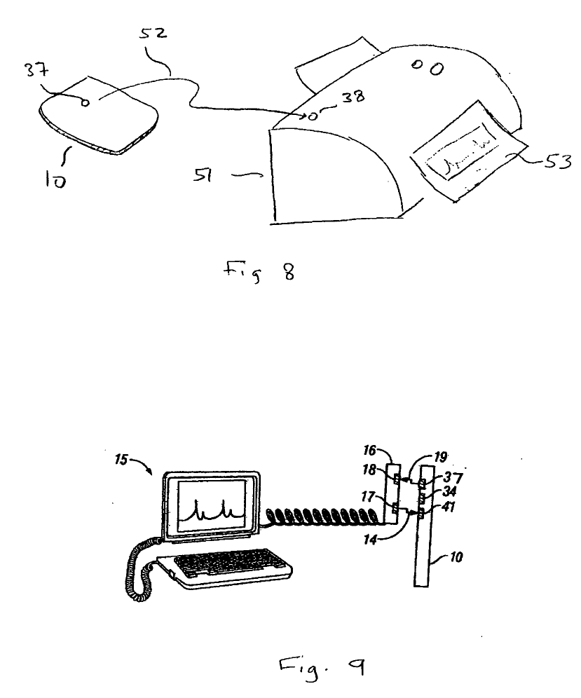

A unique feature of the present invention is the wireless transmission of preformatted report to a reporting device such as a printer or a wireless network. This allows for generation of a cardiac test report 53 without resorting to any specialized instruments. FIG. 8 shows the invented patch 10 having an infrared LED 37 for sending infrared signal 52 to a printer 51 for printing a cardiac report 53. Many standard printers are currently equipped with wireless sensors and respond to standard wireless protocols, such as IrDA (Infrared Data Association). An optocoupler tranceiver, incorporating an infrared LED and an optocoupler sensor, allows for bi-directional wireless communication of the patch with a reporting device. Similarly, using radio frequency (RF) transmitter (not shown), a report can be sent to a wireless printer or wireless network using standard RF protocols such as Bluetooth® and IEEE802®. With this method, a user or clinician can place the patch in proximity to a wireless reporting device for obtaining a cardiac report 53. This report is generated internally by the processor 33 and sent wirelessly, either automatically when in proximity to a reporting device, or manually by activating a switch. For example by incorporating a reed-switch in the patch (not shown), which can be activated by a magnet placed in proximity to the patch when printing or reporting is desired.

The cardiac report in this preferred embodiment is automatically generated and formatted by the processor 33 of the invented patch. Prior art reporting involves transmission of either raw ECG data or summary data for graphical formatting by a computer or microprocessor based device prior to sending to a printer or a display device. The invented patch performs the analysis and formatting of results internally and sends directly to a generic printer or a generic Internet browser such as Microsoft® Internet Explorer. In the later case, a capture screen is sent to the browser application by the invented patch. Once the capture screen is loaded, a report can then be printed or relayed to a medical monitoring station via the Internet.

The ability to generate a cardiac report wirelessly and directly to a generic reporting device, as provided by the present invention in a preferred embodiment, simplifies the delivery of heart health care services. For example, an individual suspecting a cardiac abnormality, can purchase a disposable ECG patch and generate a report using standard printer available in most homes. A report can also be generated and broadcast to a wireless network. To ensue privacy, an access code can be provided with each patch for entering into the capture screen prior to viewing, printing, or forwarding to remote monitoring station. Similarly, non-cardiac medical practice, such as primary physician, family physician, nursing center, etc. can not perform a basic cardiac test and obtain a report without resorting to any specialized instruments or training.

ECG data can also be sent to a remote location via standard trans-telephonic methods (not shown) whereby a telephone line adapter device can be used to send translate ECG reports from the patch to the telephone line. The adapter unit can communicate wirelessly to the patch via infrared or RS signals and subsequently dial the reporting center and transmit the cardiac report thereto. An ECG report may also be retrieved by an interrogation device as shown in FIG. 9 (not to scale). In this example, optical signal 19 representing ECG data from an infrared LED 37 incorporated within the disposable patch 10 is sent to an optical receiver 18 incorporated in the interrogation wand 16 of the external interrogation device 15. The activation of the data transmission is preferably automatic. For example, a magnetic field 14 from a magnet 17 within the interface 16 triggers an activation sensor 41, i.e. a reed-switch, to initiate the ECG data transmission. Activation can also be by manual means, such as by pressing an electromechanical switch (not shown) incorporated onto the flexible substrate 20.

The wireless transmission of cardiac data may be accomplished in numerous ways and methods known in the field of medical devices and wireless data transmission. This includes optical means as shown above, radio frequency (RF), magnetic, ultrasonic, and acoustic transmission. Inductive coupling through a coil (not shown) can also be used to transmit data, as well as for powering the patch externally during the transmission.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A disposable patch for non-invasive long term continuous monitoring of a patient's ECG, comprising:
a self adhering surface of said patch for securing said patch to a selected location on the body of a patient being evaluated for a heart abnormality;
a battery;
at least two electrodes for contacting the patient's skin at said location to receive surface ECG signals, when said patch is secured at said location;
an amplifier electrically coupled to said electrodes for amplifying said received ECG signals;
a signal processor responsive to the amplified ECG signals for detecting one or more cardiac events therefrom;
a memory coupled to said processor for recording the patient's ECG at least during each of the detected cardiac events;
waterproofing to seal said patch against penetration of fluids to electrical components thereof to allow said patch to be worn continuously by the patient for at least 14 days including during bathing; and
a transmitter for wireless transmission of the recorded ECG to a remote reporting device.

2. The patch of claim 1, wherein said battery has sufficient capacity to power the electrical components of said patch continuously for at least 30 days.

3. The patch of claim 1 further comprising a manually operable switch to mark a cardiac event experienced by the patient when wearing said patch.

4. The patch of claim 1 further comprising an indicator for producing an alert to one of the patient's senses in response to a cardiac event detected by said processor.

5. The patch of claim 1, wherein said patch is relatively flexible to adapt to the contour of the patient's body at said secured location, and relatively flat with a maximum thickness less than about 3 mm.

6. The patch of claim 1 wherein said signal processor is adapted to detect a cardiac event including arrhythmia, syncope, block, palpitation, transient ischemic attack, myocardial infarction and fibrillation.

7. The patch of claim 1, further comprising a flexible electrical circuit for interconnecting electrical components within said patch to perform their respective functions.

8. The patch of claim 1, wherein said processor is adapted to perform at least one of signal averaging and segment analysis.

9. The patch of claim 1, wherein said patch comprises multiple electrodes sufficient to enable said processor to perform diagnostic analysis.

10. The patch of claim 1, wherein said waterproofing comprise a waterproof adhesive on the perimeter of an inside surface of the patch.

11. The patch of claim 1, further comprising an anti-microbial agent on said patch to prevent contamination thereof during extended wear of said patch.

12. The patch of claim 1, further comprising air permeable material to allow air access to the skin and prevent accumulation of moisture thereon.

13. The patch of claim 1, wherein said transmitter includes means for transmitting ECG of cardiac events via telephone.

14. A patch for non-invasive monitoring of ECG signals, comprising:
means for self adhering said patch to the chest of a person being evaluated for a heart abnormality;
at least two electrodes for contacting said person's skin surface, said electrodes receiving the surface potential ECG signals;
an amplifier for amplifying said ECG signals from said electrodes;
a processor for performing analysis of said amplified ECG signals and producing a preformatted report;
a battery for powering said patch; and
means for wireless transmission of said preformatted report to a reporting device.

15. The patch of claim 14, wherein said reporting device is any of a printer and a wireless network.

16. The patch of claim 14, wherein said wireless transmission comprises any of infrared (IR) signals and radio frequency (RF) signals.

17. The patch of claim 14 further comprising an indicator to alert the person to a cardiac event detected by said processor.

18. The patch of claim 14 further comprising a switch to mark a cardiac event experienced by said person wearing said patch.

19. The patch of claim 14, wherein said patch comprises multi-lead electrodes for performing diagnostic analysis.

20. The patch of claim 14, wherein said patch is “C” shaped encompassing the person's left breast.

21. The patch of claim 14, wherein said patch is waterproof to allow continuous long-term wear and operation including during water exposure.

22. The patch of claim 14 further comprising means for transmitting ECG of cardiac events via the telephone.

23. A disposable patch for non-invasive monitoring of ECG comprising:
means for self adhering said patch to a person's torso, said person is being evaluated for a heart abnormality;
at least two electrodes for contacting said person's skin surface, said electrodes receiving the surface potential ECG signals;
an amplifier for amplifying said ECG signals from said electrodes;
a processor for detecting a cardiac event;
a battery for powering said patch;
an indicator for indicating the occurrence of a cardiac event detected by said processor; and
means for waterproofing said patch whereby it can be worn continuously for at least 14 days including during bathing.
